(19) United States
(12) Patent Application Publication
(10) Pub. No.: US 2015/0216919 A1
(43) Pub. Date: Aug. 6, 2015

(54) METHOD FOR PRODUCING ROASTED RED GINSENG AND ROASTED RED GINSENG PRODUCED BY SAME

(71) Applicant: Su-Gyum KIM, Chungcheingnam-do (KR)

(72) Inventors: Su-Gyum Kim, Dangjin-gu (KR); Jae-Eul Choi, Seo-gu (KR)

(21) Appl. No.: 14/427,755

(22) PCT Filed: Aug. 7, 2013
(86) PCT No.: PCT/KR2013/007110

§ 371 Date: Mar. 12, 2015

(30) Foreign Application Priority Data
Sep. 12, 2012 (KR) 10-2012-0100903

(51) Int. Cl. A61K 36/258

(52) U.S. Cl. CPC A61K 36/258

(57) ABSTRACT

The present invention relates to a method for producing roasted red ginseng, having increased contents of a specific ginsenoside and sugar as compared with unroasted ginseng, which comprises the steps of: primarily roasting a fresh ginseng at 120-230° C. for 20-40 minutes by introducing the fresh ginseng into an oven; and secondarily roasting the primarily roasted red ginseng at 150-250° C. for 20-40 minutes under the pressure of 0.01-0.1 kgf/cm², and to a roasted red ginseng produced by the said method.

### TECHNICAL FIELD

The present invention relates to a method for producing roasted red ginseng having increased contents of a specific ginsenoside and sugar as compared with unroasted ginseng, which comprises the steps of: primarily roasting a fresh ginseng at 120-230° C. for 20-40 minutes by introducing the fresh ginseng into an oven; and secondarily roasting the primarily roasted red ginseng at 150-250° C. for 20-40 minutes under the pressure of 0.01-0.1 kgf/cm², and to a roasted red ginseng produced by the said method.

### BACKGROUND ART

Since ancient times, ginseng has been recognized its pharmacological activity which is exceptional in Chinese medicine. In 488-496, Dohongkvung in Yang Nation times of China discloses in Shennong Ben Cao Jing that ginseng is mainly used as a medicine for raising the stamina of the five viscera (liver, heart, lung, kidney and spleen) and that ginseng also promotes mental stability, removes the had aura that causes diseases from entering the wrong way, makes the eye more bright, makes the brain more intelligent, makes the body lighter and lives a long life if taken for a long time.

In addition, “Myoungeuibyolrok” and “Compendium or Materia Medica”, etc. relatively extensively described the efficacies of ginseng. At the present day, many clinical studies on ginseng are conducted by eminent scholars from every country. Ginseng has been found to have numerous pharmacological activities such as increased immunity, anti-cancer, prevention and treatment of cardiovascular diseases.

Ginseng is classified into a fresh ginseng, a white ginseng, a dried ginseng, a red ginseng, etc., depending on the processing method. The fresh ginseng refers to a ginseng which dug up from the field and is not processed. The dried ginseng refers to a ginseng dried in an intact form without removing its peel. The white ginseng refers to a ginseng dried after removing the peel of the fresh ginseng.

Ginseng saponin is known as the main pharmacological ingredient of Korean ginseng and is called. “ginsenoside”. The ginseng saponin has a particular chemical structure which is different from saponin found in the other plant. The efficacy thereof is also known to be very exceptional. The chemical structures of 34 kinds of ginseng saponins have been found by the recent separation and analysis technique up to now. For example, Korean ginseng has all the 34 kinds of ginseng saponins; Chinese ginseng (Samchil) has 15 kinds of ginseng saponins; and American ginseng has 14 kinds of ginseng saponins.

Why Korean ginseng exhibited greatly excellent efficacies as compared with ginsengs of other countries has been identified scientifically.

Various forms of processed foods using the efficacy of ginseng have been developed and used, but they have problems that it is reluctance to eat depending on persons because the distinctive bitter taste and fragrance of ginseng are strong. The processing method that can lead to a synergistic effect of the active ingredients of ginseng has not been yet established. as a regular process, and the conditions for production differ by manufacturer. Therefore, there are differences in the quality of ginseng.

Korean Patent Registration No. 0471340 discloses a method for processing ginseng, and Korean Patent Registration No. 0217923 discloses a method for processing ginseng and a processed ginseng produced by the said method, but these methods are different from the method for producing a roasted ginseng of the present invention.

### DISCLOSURE OF INVENTION

### Technical Problem

The present invention have been designed to meet the requirements as described above and the object of the present invention is to provide a method for producing a roasted red ginseng which can, by subjecting to dry heat treatment as a high temperature applying a suitable pressure to the fresh ginseng, improve qualities such as sweetness, fragrance, chewing property and the like while preventing elution of the ingredients or dropping of a lateral root or a rootlet during the processing, and also increases the ginsenoside content and sugar content while reducing both the production time of a dried ginseng and the deterioration occurred during the drying.

### Technical Solution to Problem

In order to solve the above mentioned problems, the present invention provides a method for producing roasted red ginseng having increased contents of a specific ginsenoside and sugar as compared with unroasted ginseng, which comprises the steps of: primarily roasting a fresh ginseng at 120-230° C. for 20-40 minutes by introducing the fresh ginseng into an oven; and secondarily roasting the primarily roasted red ginseng at 150-250° C. for 20-40 minutes under the pressure of 0.01-0.1 kgf/cm².

The present invention provides a roasted red ginseng having improved contents of a specific ginsenoside and sugar as compared with unroasted ginseng, produced by the said method.

### Advantageous Effects of the Invention

The roasted red ginseng produced by the method of the present invention has the following effects.

1) The roasted red ginseng of the present invention can facilitate the saccharification of ginseng starch by the high temperature treatment of ginseng, and can also increase the sugar content of red ginseng up to 20 Brix, thereby reducing the bitter taste which children and foreigners dislike, and also increasing fragrance and chewing properties as well as flavor preference.

2) The steam heat treatment of a conventional ginseng has a problem where the ginsenosides are eluted by steam or water. Other methods such as hot air drying has a problem where the effective ingredients of ginseng are destroyed due to exposure to heat for a long time. However, the method according to the present invention can prevent the leakage and destruction of ginseng ingredients from the wound site by dry-heating the wound site generated during dropping of rootlet and washing process, at a high temperature while adding a suitable pressure within a short time.

3) The red ginseng roasted. by the method. of the present invention can significantly increase the content of specific ginsenosides, Rb1, Rb2, Rc, Re, Rg2, Rg3 and Rh1, as compared with the red ginseng which is roasted under the other treatment conditions, thereby providing consumers with the health directed red ginseng products that are differentiated from traditional red ginseng products.

4) The roasted ginseng of the present invention can prevent the red ginseng from cracking or a lateral root or a rootlet from dropping during the processing into red ginseng, thus increasing marketability, allowing mass production within. a short period of time and also reducing production costs. In addition, the effects of sterilization in the course of roasting ginseng can facilitate the distribution of ginseng, store red ginseng in one root form for a long time and allow the long-term storage by dry-freezing and be suitably used as a raw material for food.

### BEST MODE FOR THE INVENTION

In order to achieve the object of the present invention, there is provided a method for producing roasted red ginseng having increased contents of specific ginsenoside and sugar as compared with unroasted ginseng, which comprises the steps of: primarily roasting a fresh ginseng at 120-230° C. for 20-40 minutes by introducing the fresh ginseng into an oven; and secondarily roasting the primarily roasted red ginseng at 150-250° C. for 20-40 minutes under the pressure of 0.01-0.1 kgf/cm²

In the method for producing the roasted red ginseng according to the present invention, the specific ginsenoside may be one or more selected from the group consisting of Rb1, Rb2, Rc, Re, Rg2, Rg3 and Rh1, but are not limited thereto.

Also, in the method for producing roasted red ginseng according to the present invention, the sugar content may be 18 to 20 Brix, but is not limited thereto.

Specifically, the method for producing the roasted. red ginseng according to the present invention comprises the steps of: primarily roasting a fresh ginseng at 120-160° C. for 30 minutes by introducing the fresh ginseng into an oven; and secondarily roasting the primarily roasted red ginseng at 150-187° C. for 30 minutes under the pressure of 0.01 kgf/cm².

In the method for producing the roasted red ginseng according to the present invention, it is preferable to roast the ginseng with an oven which can be sealed upon roasting of the ginseng. The sealed oven can shorten the time of temperature rise and keep the temperature for sterilization of red ginseng. When the ginseng is exposed, to a heat for a long time with a steam heat or a hot air drying without using art oven, elution of ingredients or heat damage may occur, leading to problems such as a change of color and gloss, a shrinkage and a destruction of nutrients. Further, the sterilization is not made and so a long-term storage is not possible. Therefore, it is preferable to apply a heat within 1 hour using the oven.

In this regard, the roasting conditions as described above can increase the contents of specific ginsenoside and sugar and reduce the bitter taste while not reducing the quality of ginseng, and additionally process the ginseng in large quantities within a short time, thus processing the ginseng economically. In addition, the roasted ginseng of the present ginseng is completely sterilized, and has no secondary infection and thus long-term storage is possible. However, roasting the ginseng while deviating the above conditions, will reduce the quality of ginseng, slightly increase the specific ginsenoside and sugar contents, and not completely undergo sterilization.

The present invention also provides a roasted red ginseng prepared by the above method. The roasted red ginseng also includes a dried one. The roasted red ginseng of the present invention has the increased contents of specific ginsenoside and sugar and so can be taken in an intact form. The present roasted red ginseng can be used as a raw material for food. The products which can use the above roasted red ginseng as a raw material may include a red ginseng extract and pouch. The kind of food include, but is not limited to, candy, confectionery, chocolate, beverage, biscuit and the like.

Hereinafter, the present invention will be described in detail by way of examples. However, the following examples are only intended to illustrate the invention, but not to limit the context of the present invention.

### PRODUCTION EXAMPLE 1

### Production of Roasted Red Ginseng

The fresh ginseng was introduced into an oven and then primarily roasted at a temperature of 120-160° C. for 30 minutes. The primarily roasted red ginseng was secondarily roasted at a temperature of 150-187° C. for 30 minutes under the pressure of 0.01 kgf/cm².

### EXAMPLE 1

### Ginsenoside Content of the Roasted Red Ginseng According to the Roasting Conditions

The ginsenoside content of the roasted red ginseng produced by changing the roasting conditions shown in Table 1 below.

TABLE 1
Ginsenoside content of the roasted red ginseng according to the roasting conditions

| | Pressure | | | | | | | | | | | | | |
|---|---|---|---|---|---|---|---|---|---|---|---|---|---|---|
| Class | (kgf/cm²) | Rb1 | Rb2 | Rb3 | Rc | Rd | Re | Rf | Rg1 | Rg2 | Rg3 | Rh1 | Rh2 | Total |
| Fresh ginseng | No* | 1.64 | 0.25 | 0.04 | 0.38 | 0.05 | 2.15 | 0.36 | 1.35 | 0.06 | 0.00 | 0.07 | 0.00 | 5.53 |
| 160° C. 5 min | No | 0.89 | 0.29 | 0.06 | 0.42 | 0.13 | 1.87 | 0.33 | 1.66 | 0.03 | 0.01 | 0.09 | 0.00 | 5.79 |
| 200° C. 20 min | No | 1.10 | 0.24 | 0.12 | 0.68 | 0.02 | 1.96 | 0.80 | 1.95 | 0.06 | 0.00 | 0.43. | 0.00 | 7.36 |
| 200° C. 30 min | No | 1.05 | 0.33 | 0.08 | 0.59 | 0.06 | 2.01 | 0.48 | 2.46 | 0.01 | 0.01 | 0.23 | 0.00 | 7.31 |
| 230° C. 30 min | No | 1.50 | 0.60 | 0.12 | 0.86 | 0.12 | 2.01 | 0.67 | 2.20 | 0.13 | 0.00 | 0.11 | 0.00 | 8.32 |
| 120~160° C. 30 min (1^{st}) | No | 1.95 | 0.68 | 0.10 | 1.06 | 0.12 | 2.72 | 0.66 | 1.78 | 0.24 | 0.02 | 0.30 | 0.00 | 9.61 |
| 150~187° C. 30 min (2^{nd}) | 0.01 | | | | | | | | | | | | | |

No*: normal pressure without applying pressure (1 atm)

As a result, it can be confirmed that the roasted red ginseng generally exhibits an increased ginsenoside content as compared to the fresh ginseng; particularly the red ginseng roasted by the method of the invention (120˜160° C. for 30 minutes (1st step), 150˜187° C. for 30 minutes, 0.01 kgf/cm² (2nd step)) exhibits the maximum contents of Rb1, Rb2, Rc, Re, Rg2 , Rg3, Rh1, and total ginsenoside as compared to the other treatment groups.

### EXAMPLE 2

### Sugar Content of the Roasted Red Ginseng According to the Roasting Conditions

The sugar content of the roasted red ginseng produced by changing the roasting condions is shown in Table 2 below.

TABLE 2
Sugar content of the roasted red ginseng
according to the roasting conditions

| | | | Pressure | Sugar content |
|---|---|---|---|---|
| Heating times | Temperature | Time | (kgf/cm²) | (Brix) |
| Fresh ginseng | — | — | — | 4.0 |
| 1st | 170~197° C. | 30 min | 0.01 | 6.6 |
| 1st | 150~184° C. | 30 min | No* | 12.5 |
| 1st | 130~162° C. | 30 min | No | 12.8 |
| 1st | 120~160° C. | 30 min | No | 19 |
| 2nd | 150~187° C. | 30 min | 0.01 | |

No*: normal pressure without applying pressure (1 atm)

As a result, it can be confirmed that the roasted red ginseng exhibits an increased sugar content as compared with the fresh ginseng; particularly, the red ginseng roasted by the method of the invention (120˜160° C. for 30 minutes (1st step), 150˜187° C. for 30 minutes, 0.01 kgf/cm² (2nd step)) exhibits the maximum sugar content as 19 Brix as compared to the other treatment groups. The increase in the sugar content is considered to reduce a bitter taste of red ginseng, thus being preferable for children and foreigner who dislike the red ginseng.

1. A method for producing roasted red ginseng having increased contents of a specific ginsenoside and sugar as compared with unroasted ginseng, which comprises the steps of: primarily roasting a fresh ginseng at 120-230° C. for 20-40 minutes by introducing the fresh ginseng into an oven; and secondarily roasting the primarily roasted red ginseng at 150-250° C. for 20-40 minutes under the pressure of 0.01-0.1kgf/cm²,

2. The method for producing roasted red ginseng having increased contents of a specific ginsenoside and sugar compared with unroasted ginseng according to claim 1 wherein the specific ginsenoside is one or more selected from the group consisting of Rb1, Rb2, Rc, Re, Rg2, Rg3 and Rh1.

3. The method for producing roasted red ginseng having increased contents of a specific ginsenoside and sugar as compared with unroasted ginseng according to claim 1 wherein the sugar content is 18˜20 Brix.

4. A roasted red ginseng having increased contents of a specific ginsenoside and sugar as compared with unroasted ginseng, produced by the method of claim 1.

5. A red ginseng-processed food produced by using the roasted red ginseng having increased contents of a specific ginsenoside and sugar according to claim 4.
